(19) United States
(12) Patent Application Publication
(10) Pub. No.: US 2020/0306244 A1
(43) Pub. Date: Oct. 1, 2020

(54) WNT INHIBITORS FOR USE IN THE TREATMENT OF FIBROSIS

(71) Applicant: NOVARTIS AG, Basel (CH)

(72) Inventors: Jennifer Leslie HARRIS, San Diego, CA (US); Peter GERGELY, Basel (CH); Jun LIU, San Diego, CA (US); Eric SVENSSON, Cambridge, MA (US)

(73) Assignee: Novartis AG, Basel (CH)

(21) Appl. No.: 16/311,366

(22) PCT Filed: Jun. 20, 2017
(86) PCT No.: PCT/IB2017/053651

§ 371 Date: Dec. 19, 2018

Related U.S. Application Data
(60) Provisional application No. 62/353,098, filed on Jun. 22, 2016.

(51) Int. Cl. A61K 31/497; C07K 16/28; A61K 9/00; A61P 13/12; A61P 17/00

(52) U.S. Cl. CPC A61K 31/497; C07K 16/2863; A61P 17/00; A61P 13/12; A61K 9/0053; A61K 9/0019

(57) ABSTRACT

The present disclosure relates to a Wingless-type (wnt) inhibitor of formula (I) for use in the treatment of fibrosis and some fibrosis mediated disorders such as stiff skin syndrome and systemic sclerosis. The present disclosure also provides a method for the treatment of fibrosis, a pharmaceutical combination comprising a wnt inhibitor of formula (I) and a second active ingredient for use in the treatment of fibrosis and also the use of a wnt inhibitor of formula (I) or a pharmaceutically acceptable salt thereof for the manufacture of a medicament for the treatment of fibrosis and fibrosis mediated disorders.

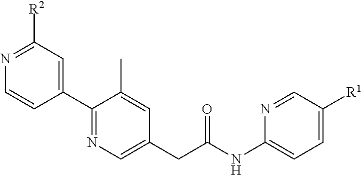

### FIELD OF THE DISCLOSURE

The present disclosure relates to the field of pharmacy, particularly to a wnt inhibitor for use in a specific indication. Specifically, the disclosure relates to a wnt inhibitor of formula (I) for use in the treatment of a disease, a method for the treatment of a disease that involves administering the wnt inhibitor of formula (I), a pharmaceutical combination comprising a wnt inhibitor of formula (I) and a second active ingredient and the use of a wnt inhibitor of formula (I) or a pharmaceutically acceptable salt thereof for the manufacture of a medicament for the treatment.

### BACKGROUND OF THE DISCLOSURE

The Wnt (Wingless) family is a group of highly conserved secreted proteins that regulate cell-to-cell interactions during embryogenesis and is implicated in carcinogenesis, aging, and fibrosis. The wnt gene was identified as an oncogene in murine mammary tumors 30 years ago and confirmed to be a key oncogenic pathway in many studies. The Wnt gene family encodes a large class of secreted proteins related to the Int1/Wnt1 proto-oncogene and Drosophila wingless (“Wg”), a Drosophila Wnt1 homologue (Cadigan et al. Genes & Development 1997, 11, 3286-3305). Wnts are expressed in a variety of tissues and organs and play a major role in many developmental processes, including segmentation in

Drosophila; endoderm development in C. elegans; and establishment of limb polarity, neural crest differentiation, kidney morphogenesis, sex determination, and brain development in mammals (Parr et al. Curr. Opinion Genetics & Devel. 1994, 4, 523-528). The Wnt pathway is a master regulator in animal development, both during embryogenesis and in the mature organism (Eastman et al. Curr. Opin. Cell Biol. 1999, 11, 233-240; Peifer et al. Science 2000, 287, 1606-1609).

Wnt signals are transduced by the Frizzled (“Fzd”) family of seven transmembrane domain receptors (Bhanot et al. Nature 1996, 382, 225-230). Wnt ligands bind to Fzd, and in so doing, activate the cytoplasmic protein Disheveled (Dvl-1, 2 and 3 in humans and mice) (Boutros et al. Mech. Dev. 1999, 83, 27-37) and phosphorylate LRPS/6. A signal is thereby generated which prevents the phosphorylation and degradation of Armadillo/β(beta)-catenin, in turn leading to the stabilization of β-catenin (Perrimon et al., Cell 1994, 76, 781-784). This stabilization is occasioned by Dvl's association with axin (Zeng et al. Cell 1997, 90:181-192), a scaffolding protein that brings various proteins together, including GSK3, APC, CK1, and β-catenin, to form the β-catenin destruction complex.

Fibro-proliferative responses are a necessary part of normal wound healing after injury. However, uncontrolled persistence of this response can lead to the excessive deposition of extracellular matrix that ultimately results in fibrosis with the loss of organ function. Fibrosis, the formation of excessive amounts of fibrotic or scar tissues, is a common pathologic problem in medicine. Scar tissue, occludes arteries, immobilizes joints and damaged internal organs, wreaking havoc on the body's ability to maintain vital functions. Fibrosis can follow surgery in the form of adhesions, keloid tumors, or hypertrophic (very severe) scarring. Fibrosis causes contractures and joint dislocation following severe bums, wounds, or orthopaedic injuries; it can occur in any organ is the sequelae to many disease states, such as hepatitis (liver cirrhosis), hypertension (heart failure), tuberculosis (pulmonary fibrosis), scleroderma (fibrotic skin and internal organs), diabetes (nephropathy), and atherosclerosis (fibrotic blood vessels). Fibrotic growth can also proliferate and invade the healthy tissue that surrounds it even after the original injury heals. In most cases, fibrosis is a reactive process, and several different factors can apparently modulate the pathways leading to tissue fibrosis. Such factors include the early inflammatory responses, local increase in fibroblast cell populations, modulation of the synthetic function of fibroblasts, and altered regulation of the biosynthesis and degradation of collagen.

Fibrosis is the final, common pathological outcome of many chronic inflammatory diseases. Fibrosis is defined by the excessive accumulation of fibrous connective tissue (components of the extracellular matrix (ECM) such as collagen and fibronectin) in and around inflamed or damaged tissue, which can lead to permanent scarring, organ malfunction and, ultimately, death, as seen in end-stage liver disease, kidney disease, idiopathic pulmonary fibrosis and heart failure. Fibrosis is also a major pathological feature of many chronic autoimmune diseases, including scleroderma, rheumatoid arthritis, Crohn's disease, ulcerative colitis, myelofibrosis and systemic lupus erythematosus. Fibrosis also influences tumor invasion and metastasis, chronic graft rejection and the pathogenesis of many progressive myopathies. It is a highly heterogeneous in its clinical and autoimmune manifestations (e.g. idiopathic pulmonary fibrosis, stiff skin syndrome, systemic sclerosis) requiring an individualized therapy.

First described by Esterly and McKusick (Esterly et al. Pediatrics 1971, 47, 360-369) in 1971, stiff skin syndrome (SSS) is a rare congenital condition associated with striking scleroderma-like changes in the skin and it is characterized by hard, thick skin, usually over the entire body, which limits joint mobility and causes flexion contractures. Other occasional findings include focal lipodystrophy and muscle weakness. Domain-specific mutations in the Fibrillin-1 gene and the consequent perturbation of both microfibrillar assembly and microfibril-integrin interactions contribute in part to the pathogenesis of stiff skin syndrome through dysregulation of TGF-β signaling (Loeys et al. Science Trans. Med. 2010, 2, 1-10).

Systemic sclerosis (SSc) is a common and etiologically mysterious form of scleroderma. SSc affects for example about 1 in 5,000 individuals in the United States. Familial recurrence is extremely rare, and causal genes have not been identified. While the onset of fibrosis in SSc typically correlates with the production of autoantibodies, whether they contribute to disease pathogenesis or simply serve as a marker of disease remains controversial, and the mechanism for antibody induction is largely unknown.

Fibrosis of the skin and internal organs is a key feature of systemic sclerosis (SSc). Since fibrosis can disrupt the physiological tissue architecture and lead to organ failure, it causes much of the morbidity and mortality in patients with systemic sclerosis (SSc). In particular, Systemic sclerosis (SSc) is a prototypical idiopathic systemic fibrotic disease that affects the skin and several internal organs such as lungs, heart, gastrointestinal tract and kidneys. Similar to other fibrotic diseases, failure of the affected organs is common and results in high morbidity and significantly increased mortality. Some preliminary studies on rodent were conducted in the recent years in order to understand the diseases pathways. In this context, appropriate in vivo models are available that reflect the pathogenesis and mimic complex disease processes of SSc. Several murine and avian models are available to study different aspects of the disease.

The model of bleomycin-induced skin fibrosis is widely used in SSc research (Beyer C. et al. Arthritis and Rheumatism. 2010, 62, 2831-2844). The model of bleomycin-induced skin fibrosis mimics inflammatory changes in SSc that often occur early in the disease course. Bleomycin treatment induces the production of reactive oxygen species, causes damage to endothelial cells and other cell types, and leads to the expression of adhesion molecules. This attracts leukocytes, including T lymphocytes and B lymphocytes, macrophages, eosinophils, and mast cells, all of which infiltrate into lesional skin and activate resident fibroblasts. Activated fibroblasts then produce and release large amounts of ECM, which result in skin fibrosis at the site of bleomycin injection.

Another model that can be used is the TSK-1 and TSK-2 mouse models (Beyer C. et al. Arthritis and Rheumatism 2010, 62, 2831-2844). In TSK-1 mice, a tandem duplication of the fibrillin 1 gene (Fbn1) results in a characteristic phenotype with tightening of the skin (Siracusa L. D. et al. Genome Res. 1996, 6, 300-313). Fibrosis can be established by an increase of certain parameters such as collagen level (Avouac J. et al. Arthritis and rheumatism 2012, 64, 5, 1642-1652), hydroxyproline content (Woessner J. F. Arch. Biochem. Biophys. 1961, 93, 440-447), myofibroblast count (Akhmetshina A. et al. Arthritis and Rheumatism 2009, 60, 1, 219-224) and dermal thickness (Akhmetshina A. et al. FASEB J. 2008, 22, 2214-2222) compared to healthy candidates; and equally the efficacy of the candidate pharmaceutical agents is is assessed based on the reduction or even full reversal of said parameters.

Among fibrotic diseases, Systemic sclerosis (SSc) is associated with one of the highest morbidity rates, with a 10-year survival of 60-70% in the diffuse subset of patients (Nikpour, M. et al. Current opinion in rheumatology 2014, 26, 131-137). Currently, there are no disease modifying therapies for SSc, and immunomodulatory therapies such as cyclophosphamide or autologous hematopoietic stem cell transplantation have shown disappointing results in patients (Silver, K. C. et al. Rheumatic diseases clinics of North America 2015, 41, 439-457; Van Laar, J. M. et al. Jama 2014, 311, 2490-2498).

Although fibrogenesis is becoming increasingly recognized as a major cause of morbidity and mortality in most chronic inflammatory diseases, there are few, if any, treatment strategies available that specifically target the pathogenesis of fibrosis. In addition, there is no product or treatment of fibrosis yet available for the treatment of patients.

### SUMMARY OF THE DISCLOSURE

Surprisingly, it was observed that wnt inhibitors, as a sole active ingredient, could interact within the fibrosis pathway to provide a treatment option for fibrosis. The wnt inhibitor presented herein proved efficacious enough even to reverse fibrosis. In the same manner, combination comprising the wnt inhibitor and the second active ingredient can work efficiently as well.

The first aspect of the present disclosure is a wnt inhibitor of formula (I)

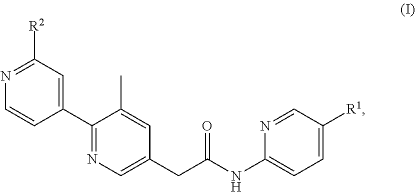

wherein R¹ is

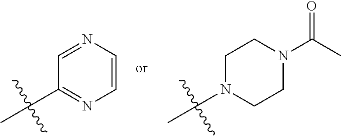

and R² is CH₃ or F, or a pharmaceutically acceptable salt thereof, for use in the treatment of fibrosis.

Another aspect of the disclosure provides a method for the treatment of fibrosis comprising administering a therapeutically effective amount of wnt inhibitor to a patient in need thereof.

A further aspect of the disclosure relates to a pharmaceutical combination comprising a wnt inhibitor of formula (I) and a second active ingredient.

A further aspect of the disclosure relates to a pharmaceutical combination comprising a wnt inhibitor of formula (I) and a second active ingredient for use as a medicine in the treatment of fibrosis.

A yet further aspect of the disclosure discloses the use of a wnt inhibitor or a pharmaceutically acceptable salt thereof for the manufacture of a medicament for the treatment of fibrosis.

### BRIEF DESCRIPTION OF FIGURES

FIG. 1A: shows that orally dosed compound of formula (I′) inhibits fibrosis in mouse bleomycin model. Bleomycin was injected in 6-week old female C57/BI6 for 3 weeks. The control group was injected with saline. The bleomycin treated animals were further dosed with or without compound of formula (I′) for another 3 weeks.

FIG. 1B: depicts that the orally dosed compound of formula (I′) inhibits fibrosis in mouse bleomycin model. The figure shows a reduction of the skin thickness in bleomycin-induced mouse fibrosis model while using compound of formula (I′) at a dose of 2.5, 5 and 10 mg/kg.

FIG. 1C: depicts that the orally dosed compound of formula (I′) inhibits fibrosis in mouse bleomycin model. The figure shows a reduction of the hydroxyproline content in bleomycin-induced mouse fibrosis model while using compound of formula (I′) at a dose of 2.5, 5 and 10 mg/kg.

FIG. 1D: shows that the orally dosed compound of formula (I′) inhibits fibrosis in mouse bleomycin model. The figure demonstrates a reduction of the myofibroblast count in bleomycin-induced mouse fibrosis model while using compound of formula (I′) at a dose of 2.5, 5 and 10 mg/kg.

FIG. 1E: depicts that the orally dosed compound of formula (I′) inhibits fibrosis in mouse bleomycin model and shows the pharmacokinetic (PK) measurements and parameters for compound of formula (I′) following the final dose.

FIG. 2A: shows that compound of formula (I′) inhibits the Wnt pathway in vivo. Bleomycin was injected in Balb/C mice, with or without orally dosed compound of formula (I′) at 5 mg/kg. The figure shows blood samples as collected at indicated time points after the last dose on day 25, plasma drug concentration and exposure were determined by LCMS.

FIG. 2B: depicts that compound of formula (I′) inhibits the Wnt pathway in vivo. Bleomycin was injected in Balb/C mice, with or without orally dosed compound of formula (I′) at 5 mg/kg. Skin tissue samples were collected 7 hour after the last dose, mRNA expression levels of Axing and Gapdh were examined with TaqMan.

FIG. 3A: shows evidence of reversal of fibrosis in Tsk-model while using compound of formula (I′) and depicts the mouse study scheme where five week old wild type or tight skin mice (Tsk-1 model) were dosed with or without compound of formula (I′) for five weeks.

FIG. 3B: shows evidence of reversal of fibrosis in Tsk-model while using compound of formula (I′) and depicts the reduction of the skin thickness in tight skin mice (Tsk-1 model) while using compound of formula (I′) at a dose of 2.5, 5 and 10 mg/kg.

FIG. 3C: shows evidence of reversal of fibrosis in Tsk-model while using compound of formula (I′) and depicts the reduction of the hydroxyproline content in tight skin mice (Tsk-1 model) while using compound of formula (I′) at a dose of 2.5, 5 and 10 mg/kg.

FIG. 3D: shows evidence of reversal of fibrosis in Tsk-model while using compound of formula (I′) and shows the reduction of the myofibroblast count in tight skin mice (Tsk-1 model) while using compound of formula (I′) a dose of 2.5, 5, 10 mg/kg.

FIG. 3E: shows evidence of reversal of fibrosis in Tsk-model while using compound of formula (I′) and depicts the pharmacokinetic (PK) measurements and parameters for compound of formula (I′) following final dose.

FIG. 3F: shows evidence of reversal of fibrosis in Tsk-model while using compound of formula (I′) and shows the Haematoxylin and eosin staining of the skin tissues samples from all dosing groups.

FIG. 4: shows evidence of reversal of fibrosis in mouse bleomycin model. The figure shows a reduction of the skin thickness in bleomycin-induced mouse fibrosis model while using compound of formula (I′) and also while using a compound of formula (I″).

### DETAILED DESCRIPTION OF THE DISCLOSURE

The present disclosure reports a new method to treat fibrosis using wnt inhibitors, as a sole active ingredient or in combination, which will interact and regulate fibroblast activation in patient with fibrosis disorders. Based on expression profiling we identified Wnt signaling to be activated in systemic sclerosis (SSc) (expression profiling of HV and SSc) and stiff skin syndrome (SSS). In addition, the use of a wnt inhibitor was found to be efficacious in rodent fibrosis model—Tsk-1 Mouse Model. The wnt inhibitor of formula (I′) showed strong efficacy also in Bleomycin-induced skin fibrosis model. In both mice model expressing fibrosis an increase of the dermal thickness, the hydroxyproline content and the myofibroblast count was observed compared to untreated or healthy mice. Treatment with the compound of the present invention as a sole active ingredient surprisingly showed a regression of fibrosis or reversal of the dermal thickness, the hydroxyproline content and the myofibroblast count towards a healthy level, therefore opening the door to an effective treatment of skin fibrosis.

According to the present disclosure, a wnt inhibitor of formula (I)

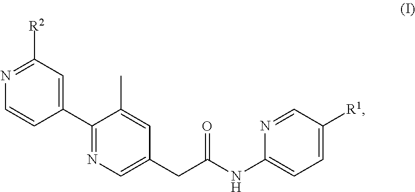

wherein R¹ is

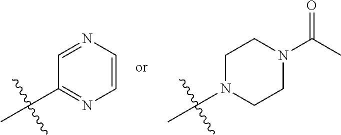

and R² is CH₃ or F, or a pharmaceutically acceptable salt thereof, can thus be used in the treatment of fibrosis.

The term “treatment” comprises, for example, the therapeutic administration of the wnt inhibitor as described herein to a warm-blooded animal, in particular a human being, in need of such treatment with the aim to cure the disease or to have an effect on disease regression or on the delay of progression of a disease. The terms “treat”, “treating” or “treatment” of any disease or disorder refers to ameliorating the disease or disorder (e.g. slowing or arresting or reducing the development of the disease or at least one of the clinical symptoms thereof), to preventing or delaying the onset or development or progression of the disease or disorder. In addition those terms refers to alleviating or ameliorating at least one physical parameter including those which may not be discernible by the patient and also to modulating the disease or disorder, either physically (e.g. stabilization of a discernible symptom), physiologically (e.g. stabilization of a physical parameter), or both.

Wnt inhibitors can be any compound that targets, decreases or inhibits the activity of the wnt signaling in a cell. The wnt inhibitors include but are not limited to the compounds disclosed in WO2010/101849. The wnt inhibitor for use in the treatment of fibrosis can be selected, for example, from the group consisting of N-(5-(4-acetylpiperazin-1-yl)pyridin-2-yl)-2-(2′-fluoro-3-methyl-2,4′-bipyridin-5-yl) acetamide, and 2-(2′,3-dimethyl-2,4′-bipyridin-5-yl)-N-(5-(pyrazin-2-yl)pyridin-2-yl)acetamide, or a pharmaceutically acceptable salt thereof.

More particularly, the wnt inhibitor can be N-(5-(4-acetylpiperazin-1-yl)pyridin-2-yl)-2-(2′-fluoro-3-methyl-[2,4′-bipyridin]-5-yl)acetamide, or a pharmaceutically acceptable salt thereof, of formula (I′)

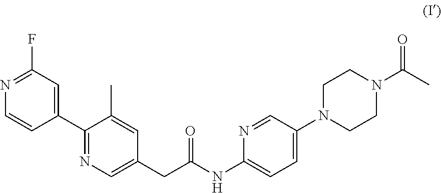 [SMILES: CC(=O)N1CCN(c2ccc(NC(=O)Cc3cnc(-c4ccnc(F)c4)c(C)c3)nc2)CC1]

as disclosed in WO2010/101849 (compound 193, example 41).

The wnt inhibitor can be 2-(2′,3-dimethyl-[2,4′-bipyridin]-5-yl)-N-(5-(pyrazin-2-yl)pyridin-2-yl)acetamide, or a pharmaceutically acceptable salt thereof, of formula (I″)

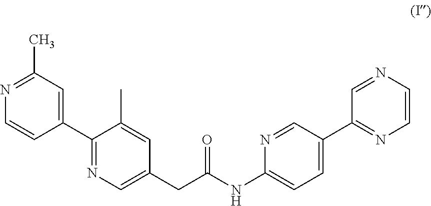 [SMILES: Cc1cc(-c2ncc(CC(=O)Nc3ccc(-c4cnccn4)cn3)cc2C)ccn1]

as disclosed in WO2010/101849 (compound 86, example 10).

The term “Pharmaceutically acceptable salts” can be formed, for example, as acid addition salts, preferably with organic or inorganic acids. Suitable inorganic acids are, for example, halogen acids, such as hydrochloric acid. Suitable organic acids are, e.g., carboxylic acids or sulfonic acids, such as fumaric acid or methanesulfonic acid. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. Any reference to the free compounds hereinbefore and hereinafter is to be understood as referring also to the corresponding salts, as appropriate and expedient. The salts of compound of formula (I) are preferably pharmaceutically acceptable salts; suitable counter-ions forming pharmaceutically acceptable salts are known in the field.

The term “pharmaceutically acceptable” refers to those compounds, materials, compositions, and/or dosage forms which are suitable for use in contact with the tissues of human beings and animals without excessive toxicity, irritation, allergic response, or other problem or complication, commensurate with a reasonable benefit/risk ratio.

Fibrosis that can be treated according to the present disclosure is a major pathological feature of many chronic autoimmune diseases, including scleroderma, rheumatoid arthritis, Crohn's disease, ulcerative colitis, myelofibrosis and systemic lupus erythematosus, cardiovascular fibrosis such as that associated with left ventricular hypertrophy, myocardial infarctions, dilated cardiomyopathy, valvular heart disease, and myocarditis. Other disease states which are fibrotic in nature and can be treated according to this disclosure are skin fibrosis, idiopathic fibrosis, pulmonary fibrosis, renal interstitial fibrosis, liver fibrosis, scleroderma, systemic sclerosis, stiff skin syndrome and idiopathic pulmonary fibrosis.

According to the present disclosure, the particularly amenable disease conditions to be treated with the aforementioned wnt inhibitors are systemic sclerosis and stiff skin syndrome. More specifically, the wnt inhibitors can be used to treat systemic sclerosis (SSc). Good results from tests with animal models of stiff skin syndrome (SSS) have been obtained and thus the wnt inhibitors can be used in the treatment of said condition as well.

In particular Stiff skin syndrome (SSS) and systemic sclerosis are conditions associated with striking scleroderma-like changes in the skin such as hard and thick skin, usually over the entire body, which limits joint mobility and causes flexion contractures, but also excessive accumulation of fibrous connective tissues (such as collagen and fibronectin) in and around inflamed or damaged tissue. The present disclosure provides an effective treatment that can reverse fibrosis by reversing parameters such as collagen level, hydroxyproline content, myofibroblast count and skin thickness.

The wnt inhibitor was shown to have sufficient efficacy to stop progression of skin fibrosis when used alone. In addition, the wnt inhibitor as described herein could even cause reversal of fibrosis. In the experiments, the reversal of fibrosis was observed and identified when looking at parameters such as dermal skin thickness, hydroxyproline levels and myofibroblast count. The parameter levels were reduced; the hydroxyproline content levels were brought back to the baseline level. Therefore, the wnt inhibitor of formula (I) can be used in the treatment of fibrosis, or any of its specific forms, such as SSc or SSS. The effect of the treatment can lead also to reversal of fibrosis.

The term “reversal” of fibrosis refers to the reduction or regression of fibrosis towards the level observed in a healthy candidate (as opposed to stopping the progression of skin thickening). Fibrosis is associated with an increase of the dermal skin thickness (1 to 3 time fold more thick than a healthy candidate), of the hydroxyproline content (1 to 2 time fold more elevated than a healthy candidate) and of the myofibroblast count (2 time fold more elevated than a healthy candidate) compared to healthy candidates. Therefore, the reversal of the fibrosis symptoms refers to alleviating or ameliorating the physical parameter associated with fibrosis towards the levels of healthy candidates. More specifically, reversal includes a regression of hydroxyproline content from about 20% to about 100% of fibrosis, more preferably from about 40% to 100% of fibrosis, more preferably from about 50% to 100% of fibrosis. The same applies to dermal skin thickness measurement. In a particular embodiment, the fibrosis is resorbed. When measuring myofibroblast count, the reversal is determined, when the count is reduced by at least 50%, preferably at least by at least 70% or more preferably the count is reduced by at least 80%. The respective parameters can be measured by the methods described in the experimental section.

Another aspect of the present disclosure provides a wnt inhibitor of formula (I) for use in the treatment of fibrosis, wherein the reversal of fibrosis is determined.

Another aspect of the present disclosure provides a method for the treatment of fibrosis comprising administering a therapeutically effective amount of wnt inhibitor of formula (I) to a patient in need thereof.

The term “patient” refers to a warm-blooded animal, in particular a human being that would benefit biologically, medically or in quality of life from the treatment. Subject or patient that can get the wnt inhibitor administered, as a sole active ingredient or as a combination, encompasses mammals and non-mammals. In a most preferred embodiment, the subject or patient is human. It may be a human who has been diagnosed as in need of treatment for a disease or disorder disclosed herein.

The term “effective amount” means the amount of the subject compound that will engender a biological or medical response in a cell, tissue, organ, system, animal or human that is being sought by the researcher, veterinarian, medical doctor or other clinician. The effective dosage of each wnt inhibitor employed according to the disclosure may vary depending on the particular compound or pharmaceutical composition employed, the mode of administration, the condition being treated, the severity the condition being treated. A physician, clinician or veterinarian of ordinary skill can readily determine and prescribe the effective amount of the drug required to prevent, counter or arrest the progress of the condition. Optimal precision in achieving concentration of drug within the range that yields efficacy requires a regimen based on the kinetics of the wnt inhibitor's availability to target sites. This involves a consideration of the distribution, equilibrium and elimination of a drug. The therapeutically effective dosage of the wnt inhibitor of the disclosure, or pharmaceutical composition, is dependent on the species of the subject, the body weight, age and individual condition, the disorder or disease or the severity thereof being treated, and can be determined by standard clinical techniques. In addition, in vitro or in vivo assays can optionally be employed to help identify optimal dosage ranges. The precise dose to be employed can also depend on the route of administration, and the seriousness of the condition being treated and can be decided according to the judgment of the practitioner and each subject's circumstances in view of, e.g., published clinical studies. The same applies to a combination comprising a wnt inhibitor and a second active ingredient.

The wnt inhibitor of formula (I) can be used in the treatment of fibrosis or in a method for the treatment of fibrosis wherein said wnt inhibitor, as described herein, can be administered in treatment cycles comprising an administration period of up to 2 months, preferably of up to 1 month, followed by a rest period of at least a week to 3 months, preferably the rest period of 1 to 4 weeks. More preferably the wnt inhibitor is administered in treatment cycles comprising an administration period of up to 1 month followed by a rest period of 4 weeks. More preferably wnt inhibitor is administered in treatment cycles comprising an administration period of up to 1 month followed by a rest period of 3 weeks. Or more preferably the wnt inhibitor is administered in treatment cycles comprising an administration period of up to 1 month followed by a rest period of 2 weeks. More preferably the wnt inhibitor is administered in treatment cycles comprising an administration period of up to 5 weeks followed by a rest period. The rest period can last at least a week to 3 months. More preferably the wnt inhibitor is administered in treatment cycles comprising an administration period of up to 3 weeks followed by a rest period of at least a week to 3 months. The rest period can be for example at least a week long. The rest period following the up to 3-week treatment cycle can last up to 3 months.

As described above, the wnt inhibitor of formula (I) can be administered as the sole active ingredient.When the wnt inhibitor is N-(5-(4-acetylpiperazin-1-yl)pyridin-2-yl)-2-(2′-fluoro-3-methyl-2,4′-bipyridin-5-yl)acetamide or a pharmaceutically acceptable salt thereof, it can be administered at a dose of 40 to 80 mg/day. When the wnt inhibitor is 2-(2′,3-dimethyl-2,4′-bipyridin-5-yl)-N-(5-(pyrazin-2-yl) pyridin-2-yl)acetamide or a pharmaceutically acceptable salt thereof, it can be administered at a dose of 5 to 50 mg/day.

The wnt inhibitor of formula (I) for use in the treatment of fibrosis or a method for the treatment of fibrosis can also be administered in combination with a second active ingredient. More specifically the second active ingredient can be an inhibitor of the TGFbeta signaling pathway. In particular, the second active ingredient can be selected from the group consisting of fresolimumab and metelimumab. More specifically the second active ingredient is the inhibitor of actavin type 2 receptor (type 2B). Yet more specifically, the second active ingredient is selected from the group consisting of ACE-031 (Acceleron/shire), LY2495655 (Lilly), PF-06252616 (Pfizer) and bimagrumab. More specifically, the second active ingredient is bimagrumab.

In humans, the transforming growth factor-beta (TGF-beta or TGF-(3) superfamily represents a diverse set of growth factors, including bone morphogenic proteins (BMPs), growth and differentiation factors (GDFs), activins, TGF-β's, nodal, and anti-mullerian hormone (AMH) (Padua et al Cell Research 2009, 19, 89-102). Most members of this family exist in variant forms, with the TGF-β cytokine consisting of three isoforms: TGF-β1, TGF-β2, and TGF-β3. The TGF-β ligands are synthesized within the cell as dimeric pro-hormones (Gray et al. Science 1990, 247, 1328-1330). Latent dimeric forms are secreted into the extracellular matrix, where they are cleaved by furins and other convertases to form active signaling molecules (Constam et al. J. Cell. Biol. 1999, 144, 139-149). Activated TGF-(3 cytokines can then signal by bringing together two pairs of receptor serine/threonine kinases, the type I and type II receptors, forming a heteromeric complex. The human genome encodes seven type I receptors (ALKs 1-7) and five type II receptors (ActR-IIa, ActR-IIB, BMPRII, AMHRII, and TβRII) that are paired in different combinations as receptor complexes for various members of the TGF-β family. The TGF-β1 ligand preferentially signals through the TβR type II receptor and the ALKS type 1 receptor. In addition to these two classes of receptors, type III receptors such as betaglycan aid the TGF-β ligands to more efficiently bind to their cognate TGF-β receptors (Shi et al. Cell 2003, 113, 685-700).

Activins are dimeric growth and differentiation factors which belong to the transforming growth factor-beta (TGF-beta) superfamily of structurally related signaling proteins. Activins signal through a heterodimeric complex of receptor serine kinases which include at least two type I (I and IB) and two type II (II and IIB, aka ACVR2A and ACVR2B) receptors. These receptors are all transmembrane proteins, composed of a ligand-binding extracellular domain with cysteine-rich region, a transmembrane domain, and a cytoplasmic domain with predicted serine/threonine specificity. Type I receptors are essential for signaling while type II receptors are required for binding ligands and for expression/recruitment of type I receptors. Type I and II receptors form a stable complex after ligand binding resulting in the phosphorylation of type I receptors by type II receptors. The activin receptor II (ActRII) is a receptor for myostatin. Research grade polyclonal and monoclonal anti-ActRIIB antibodies are known in the art, such as those made by R&D Systems®, Minn., USA.

The particularly preferred inhibitor, Bimagrumab, also known as BYM338, is a monoclonal antibody developed to bind competitively to activin receptor type II (ActRII) with greater affinity than myostatin or activin, its natural ligands. Bimagrumab is disclosed in WO 2010/125003 and the INN was published in the WHO-INN proposed list 108, 2012, vol 26, No 4, page 407-408 (also called bimagrumabum). Bimagrumab is a fully human antibody (modified IgG1, 234-235-Ala-Ala, λ2) which binds to the ligand binding domain of ActRII, thereby preventing binding and subsequent signaling of its ligands, one of which is myostatin and activin. Myostatin, a member of the transforming growth factor beta (TGF-β) superfamily, is a secreted protein that negatively regulates skeletal muscle mass in animals and humans. Bimagrumab is cross-reactive with human and mouse ActRIIA and ActRIIB and effective on human, cynomolgus, mouse and rat skeletal muscle cells. Bimagrumab binds with extremely high affinity (KD 1.7±0.3 pM) to human ActRIIB and with relatively lower affinity to human ActRIIA (KD 434±25 pM), and is formulated for intravenous (i.v.) administration. The manufacture of bimagrumab has also been described in WO2010/125003.

The skin fibrosis can also be treated by a pharmaceutical combination comprising a wnt inhibitor of formula (I) and a second active ingredient, as defined herein. Particularly the wnt inhibitor is selected from N-(5-(4-acetylpiperazin-1-yl)pyridin-2-yl)-2-(2′-fluoro-3-methyl-2,4′-bipyridin-5-yl)acetamide, or a pharmaceutically acceptable salt thereof, and 2-(2′,3-dimethyl-2,4′-bipyridin-5-yl)-N-(5-(pyrazin-2-yl)pyridin-2-yl)acetamide, or a pharmaceutically acceptable salt thereof, and the second active ingredient is selected from TGF-β signaling fresolimumab and metelimumab, and TGF-β signaling activin receptor type 2, ACE-031, LY2495655, PF-06252616 and bimagrumab. More particularly the second active ingredient is bimagrumab.

The wnt inhibitor can thus be administered together with the second active ingredient. Such combination is suitable for the treatment of fibrosis. Specifically, the combination is suitable for the treatment of SSc and SSS.

The term “pharmaceutical combination” as used herein refers to a product obtained from mixing or combining in a non-fixed combination the active ingredients, e.g. (i) wnt inhibitor, or a pharmaceutically acceptable salt thereof, and (ii) a second active ingredient as described herein separately or together. The term “non-fixed combination” means that the active ingredients, e.g. (i) wnt inhibitor, or a pharmaceutically acceptable salt thereof and (ii) a second active ingredient, are both administered separately or together, independently at the same time or separately within time intervals, wherein such administration provides therapeutically effective levels of the active ingredient in the subject in need. The latter also applies to cocktail therapy, e.g. the administration of three or more active ingredients. This term defines especially a “kit of parts” in the sense that the combination (i) wnt inhibitor, or a pharmaceutically acceptable salt thereof and (ii) a second active ingredient (and if present further one or more co-agents) as defined herein can be dosed independently of each other. Nevertheless, it is also contemplated herein that (i) wnt inhibitor, or a pharmaceutically acceptable salt thereof and (ii) a second active ingredient could be administered in a reduced dose compared to the respective doses used when the drugs are used alone. Particularly this can be advantageous in case tolerability and drug related adverse events are problematic when using the compound. Drug dose reduction is such instances could help to leave the subject, e.g. patient on the drug, while adding the combination partner. Overall, such approach bestows the clinical team with better flexibility as to the treatment options for the subject.

The term “jointly active” may mean that the compounds may be given separately or sequentially (in a chronically staggered manner, especially a sequence specific manner) in such time intervals that they preferably, in the warm-blooded animal, especially human, to be treated, and still show a (preferably synergistic) interaction (joint therapeutic effect). A joint therapeutic effect can, inter alio, be determined by following the blood levels, showing that both compounds are present in the blood of the human to be treated at least during certain time intervals, but this is not to exclude the case where the compounds are jointly active although they are not present in blood simultaneously.

Another aspect of the present disclosure provides a pharmaceutical combination comprising an amount which is jointly therapeutically effective for the treatment of fibrosis and wherein fibrosis is selected from skin fibrosis, idiopathic fibrosis, pulmonary fibrosis, renal interstitial fibrosis, liver fibrosis, scleroderma, systemic sclerosis (SSc), stiff skin syndrome (SSS), idiopathic pulmonary fibrosis. More particularly, fibrosis is systemic sclerosis. Preferably, fibrosis is stiff skin syndrome.

The present disclosure also describes the pharmaceutical combination according to the present disclosure in the form of a “kit of parts” for the combined administration. The combination can refer to either a fixed combination in one dosage unit form, or a kit of parts for the combined administration where (i) wnt inhibitor, or a pharmaceutically acceptable salt thereof, and (ii) second active ingredient, may be administered independently at the same time or separately within time intervals, especially where these time intervals allow that the combination partners show a cooperative (=joint) effect. The independent formulations or the parts of the formulation, product, or composition, can then, e.g. be administered simultaneously or chronologically staggered, that is at different time points and with equal or different time intervals for any part of the kit of parts. In the combination therapies of the disclosure, the compounds useful according to the disclosure may be manufactured and/or formulated by the same or different manufacturers. Moreover, the combination partners may be brought together into a combination therapy: (i) prior to release of the combination product to physicians (e.g. in the case of a kit comprising wnt inhibitor and the second active ingredient); (ii) by the physician themselves (or under the guidance of a physician) shortly before administration; (iii) in the patient themselves, e.g. during sequential administration of the compound of the disclosure and the other therapeutic agent. In one embodiment the effect of the combination is synergistic.

The therapeutically effective dosage of the combination of the disclosure, or pharmaceutical composition, is dependent on the species of the subject, the body weight, age and individual condition, the disorder or disease or the severity thereof being treated, and can be determined by standard clinical techniques. In addition, in vitro or in vivo assays can optionally be employed to help identify optimal dosage ranges. The precise dose to be employed can also depend on the route of administration, and the seriousness of the condition being treated and can be decided according to the judgment of the practitioner and each subject's circumstances in view of, e.g., published clinical studies. In general, satisfactory results are indicated to be obtained systematically at daily dosages from 40 mg to 80 mg of N-(5-(4-acetylpiperazin-1-yl)pyridin-2-yl)-2-(2′-fluoro-3-methyl-2,4′-bipyridin-5-yl)acetamide, or a pharmaceutically acceptable salt thereof, orally. In general, satisfactory results are indicated to be obtained systematically at daily dosages from 5 mg to 50 mg of 2-(2′,3-dimethyl-2,4′-bipyridin-5-yl)-N-(5-(pyrazin-2-yl)pyridin-2-yl)acetamide, or a pharmaceutically acceptable salt thereof, orally. For example the wnt inhibitor can be combined in the usual daily dose. In some cases, the daily dose of wnt inhibitor can also be adjusted.

The therapeutically effective dosage of the combination as described in the present disclosure can be obtained by administrating of an anti-ActRII antibody, e.g. bimagrumab, in a dosage range from 1-10 mg/kg of the host body weight. More particularly, dosages include about 1 mg/kg body weight or about 3 mg/kg body weight or about 10 mg/kg body weight, preferably once every four weeks. Such administration is preferably carried out intravenously. Alternatively, administration is carried out subcutaneously. Another aspect of the invention provides that the combination partner anti-ActRII antibody, e.g. bimagrumab, is administered at a flat dose from 70 mg to 700 mg of active substance. More particularly, the dosage includes about 70 mg or 201 mg or 301 mg or 700 mg flat dose in subcutaneously injection, preferably once every eight weeks, preferably once every four weeks.

It was shown that the combination of the present disclosure possesses beneficial therapeutic properties, e.g. synergistic interaction, strong in-vivo and in-vitro antitumor response, which can be used as a medicine. Its characteristics render it particularly useful for the treatment of fibrosis. In particular the present disclosure provides the use of a wnt inhibitor or a pharmaceutically acceptable salt thereof for the manufacture of a medicament for the treatment of fibrosis.

### ABBREVIATIONS

ACPI atmospheric pressure chemical ionization
ActRII activin receptor type II
ALK anaplastic lymphoma kinase
AMH anti-mullerian hormone
b.i.d “Bis in die” or twice a day
Bleo Bleomycin
BMPs including bone morphogenic proteins
Dvl cytoplasmic protein Disheveled
ECM extracellular matrix
Fzd Frizzled
GDF growth and differentiation factors
HPLC High performance liquid chromatography
IgG Immunoglobulin G
IPF idiopathic pulmonary fibrosis
LC/MS Liquid Chromatography/Mass Spectrometry
mpk mg/kg
MRM Multiple reaction monitoring
NaCl Sodium Chloride
Pk Pharmacokinetic
SMA Smooth muscle actin
SSc systemic sclerosis
SSS Stiff skin syndrome
TGF-β transforming growth factor beta
Veh. vehicle
Wg Drosophila wingless
Wk week
Wnt Wingless

### EXAMPLES

Herein, we present data demonstrating that compounds of Formula (I′) and of Formula (I″), Porcupine inhibitors, attenuates Wnt signaling and reverses fibrosis in multiple experimental fibrosis mouse models. These studies point to the importance of the Wnt pathway as a causative molecular mechanism of skin fibrosis and provide basis for use of the compounds for treating and preventing diseases of skin fibrosis.

### Methods

### Animal Studies

All animals were housed and bred at the Vivarium at 4D Sciences (Germany). The experimental protocols were in compliance with animal welfare regulations and approved by the IACUC committee at GNF and the animal welfare committees at 4D Sciences.

Two different mouse models of SSc were used: bleomycin-induced skin fibrosis and the tight skin (Tsk-1) model.

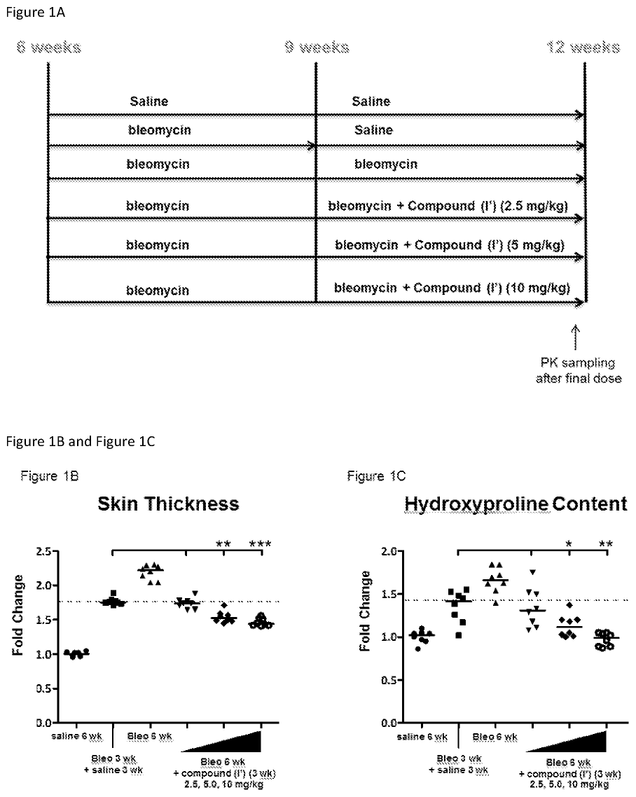

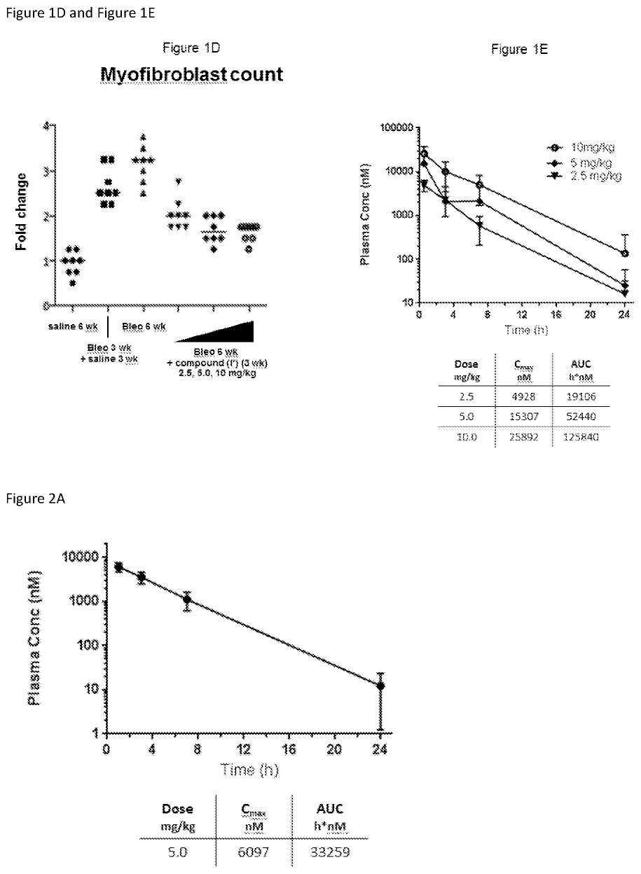

In the bleomycin model (FIG. 1 (A-E) and FIG. 2 (A-B)), fibrosis was induced in 6-week-old female C57/BI6 mice (Charles River, Sulzfeld, Germany) by injection of 100 μL 0.5 mg/ml bleomycin into a defined skin area of 1 cm' between the shoulders at the back every other day for up to six weeks. Control mice were injected with 0.9% NaCl, the solvent of bleomycin. Another group of mice received bleomycin injections for 3 weeks followed by saline (NaCl) injections for the next 3 weeks to control for spontaneous regression of fibrosis. Animals were treated with or without compound of formula (I′) at multiple dose levels twice daily for indicated weeks using suspension formulation containing 0.5% methylcellulose and 0.5% tween 80. Each group of mice were killed and the skin sections analyzed.

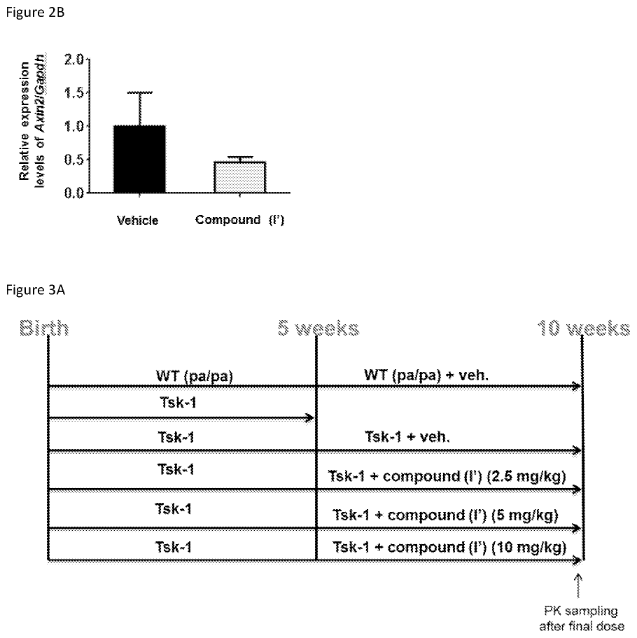

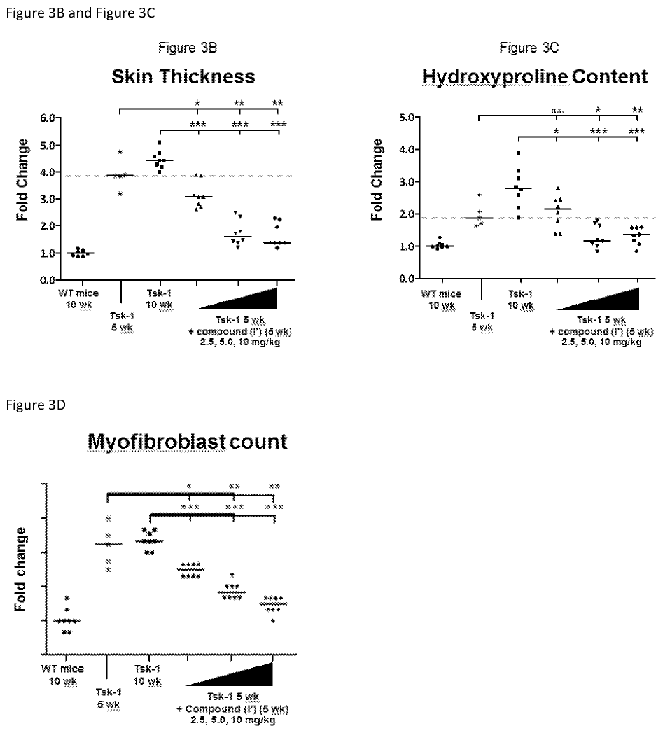

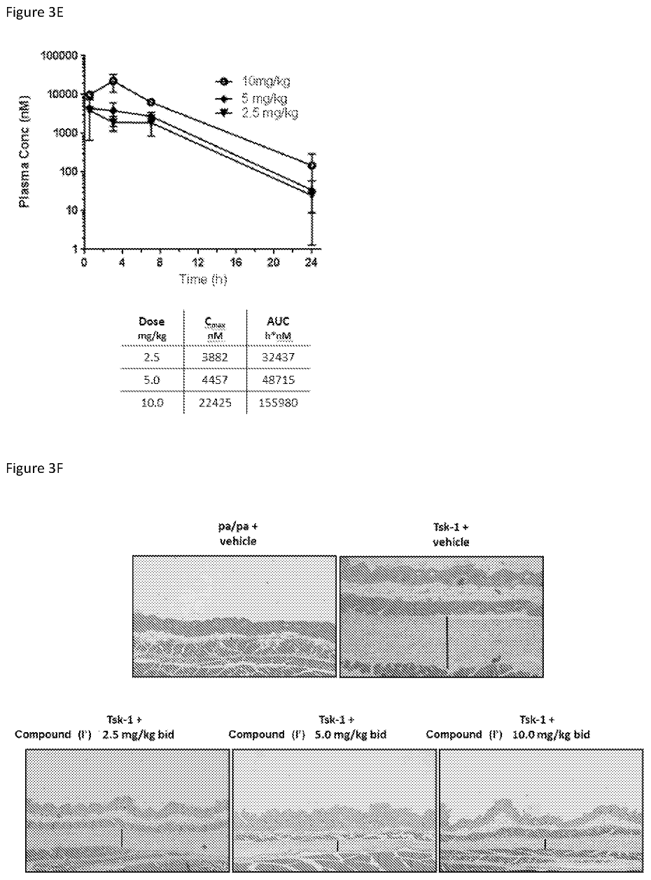

In the Tsk-1 model (FIG. 3(A-F)), treatment with compound of formula (I′) was started at the age of 5 weeks and was continued for 5 weeks. Animals were treated with or without compound of formula (I′) at multiple dose levels twice daily using suspension formulation containing 0.5% methylcellulose and 0.5% tween 80.

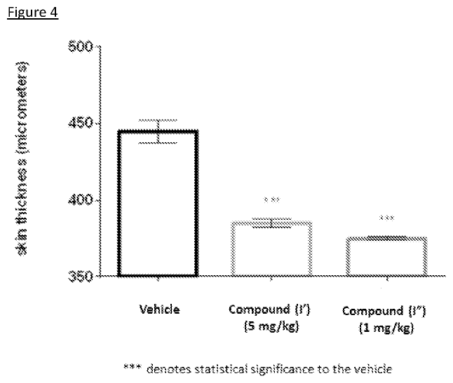

To test compound of formula (I′) and compound of formula (I″) in a mouse fibrosis model, bleomycin was injected in Balb/C mice, with or without orally dosed compound of formula (I′) (5 mg/kg b.i.d.) and compound of formula (I″) (1 mg/kg b.i.d.) for 3 weeks. Skin tissue samples were taken at the end of the study and embedded in paraffin and skin thickness was measured (FIG. 4).

### PK Analysis

The plasma concentrations of compound of formula (I′) were determined by a liquid chromatography-tandem mass spectrometry (LC/MS/MS) assay. Briefly, mouse plasma samples were extracted with a methanol-acetonitrile mixture (3:1, v/v). Supernatant was injected onto HPLC system with a Waters Atlantis T3 analytical column (2.1×30 mm, 3.5 μm, Waters Corp., Milford, Mass., USA). The mobile phases consisted of 0.1% formic acid in water (solvent A) and 0.1% formic acid in acetonitrile (solvent B), and a gradient elution method (0-1.5 min, 10% B to 95% B; 1.5-2.0 min, 95% B; 2.01 min, 10% B) at flow rate of 800 μL/min was employed for chromatographic separation. Mass spectrometric analysis was performed on a SCIEX API-4000 triple quadruple mass spectrometer (Applied Biosystems, Foster City, Calif., USA) using atmospheric pressure chemical ionization (APCI) in the positive ion mode. Multiple reaction monitoring (MRM) of compound of formula (I′) and internal standard was used for quantitative measurements, and peak integration was performed using Analyst™ 1.4 software. The lower limit of detection for the assay was 1 ng/mL. Pharmacokinetic parameters were calculated by non-compartmental regression analysis using an in house fitting program.

### RNA Extraction and RT-PCR Analysis by TaqMan

Total RNA was isolated from tissue samples and processed to perform quantitative RT-PCR analysis of Axing mRNA expression, with Gapdh serving as an internal control according to the manufacturer's instructions, as previously described.

Data were analyzed with SDS 2.0 software (Applied Biosystems), statistical analysis was conducted using PRISM.

### Histology, Hydroxyproline Contents and Myofibroblast Counts

The anti-fibrotic effects of compound of Formula (I′) on experimental skin fibrosis was evaluated by quantification of dermal or hypodermal thickening, respectively, analyses of myofibroblast counts and assessment of hydroxyproline content. Tissue samples were fixed in 10% phosphate-buffered formalin for 24 hours, then embedded in paraffin and cut into 5-μM sections. The slides were stained with hematoxylin and eosin for better visualization of the tissue structure.

The hydroxyproline content was measured according to the method as described in hydroxyproline content (Woessner J. F. Arch. Biochem. Biophys. 1691, 93, 440-447).

The dermal thickness was analyzed with a microscope by measuring the maximal distance between the epidermal-dermal junction and the dermal-subcutaneous fat junction at 4 different skin sections in each mouse.

The Hypodermal thickness in TSK-1 mice was determined by measuring the thickness of the subcutaneous connective tissue beneath the panniculus carnosus at 4 different sites of the upper back in each mouse. The evaluation was performed by 2 independent examiners.

For quantification of myofibroblasts, skin sections were deparaffinized and incubated with 5% bovine serum albumin for 60 minutes. Cells positive for α-smooth muscle actin (α-SMA) were detected by incubation with monoclonal anti-α-SMA antibodies (clone 1A4; Sigma-Aldrich, Steinheim, Germany) for 2 hours at room temperature followed by incubation with 3% hydrogen peroxide for 10 minutes. Goat anti-rabbit antibodies labeled with horseradish peroxidase were used as secondary antibodies. The expression of α-SMA was visualized with 3,3′-diaminobenzidine tetrahydrochloride. Monoclonal mouse IgG antibodies were used as controls

Data were processed with PRISM for statistical analysis.

### Results

Porcupine Inhibitor Compound of Formula (I′) in the Bleomycin-Induced Mouse Fibrosis Model To test compound of formula (I′) for anti-fibrotic effects, bleomycin was injected in 6-week-old female C57/BI6 mice for 3 weeks to induce fibrosis (FIG. 1A). The control group was injected with saline. The bleomycin treated animals were further dosed with or without compound of formula (I′) b.i.d. for another 3 weeks. As shown in FIG. 1B, compound of formula (I′) reduced dermal skin thickness at all dose levels in a dose dependent manner. Most significantly, the 5 mg/kg and 10 mg/kg groups not only stopped further fibrosis progression, but also reduced the skin thickness to levels lower than the baseline, suggesting a reversal of fibrosis. Similar observations have been obtained when examining the hydroxyproline content and myofibroblast count of mouse skin samples as shown in FIG. 1C and FIG. 1D. Compound of formula (I′) at all dose levels inhibited fibrosis and reduced the hydroxyproline content (FIG. 1C) and myofibroblast count (FIG. 1D) to levels lower than the baseline and closer to the control group (saline 6 week mice), suggesting a reversal of fibrosis, with the 5 and 10 mg/kg groups showing strong evidence of fibrosis reversal.

Plasma exposures of compound of formula (I′) were determined following the last oral dose, and PK data are shown in FIG. 1E. Compound of formula (I′) was highly absorbed and showed high oral exposures. Plasma exposures of compound of formula (I′) increased approximately dose proportionally from 2.5 mg/kg to 10 mg/kg.

Furthermore, the bleomycin treated animals were treated with or without orally dosed compound of formula (I′) at a dose of 5 mg/kg b.i.d. and compound of formula (I″) at a dose of 1 mg/kg b.i.d. for three weeks. The results shown in FIG. 4 demonstrate that both compounds induce the reduction of dermal skin thickness in such models. The compound of Formula (I″) (2-(2′,3-dimethyl-2,4′-bipyridin-5-yl)-N-(5-(pyrazin-2-yl)pyridin-2-yl)acetamide) showed even better in vivo activities compared to the compound of Formula (I′) (FIG. 4).

In a separate study to confirm the on-target activity of compound of formula (I′) in a mouse fibrosis model, bleomycin was injected in Balb/C mice, with or without orally dosed compound of formula (I′) at 5 mg/kg b.i.d. for 3 weeks. Blood samples at multiple time points after the last dose were collected. The PK analysis of the compound showed it to have good oral bioavailability (FIG. 2A). Tissue samples were collected 7 hour after the last dose, and the Wnt pathway target gene Axin2 mRNA expression level was measured with qRT-PCR, with the housekeeping gene, Gapdh, as an internal control. As shown in FIG. 2B, compound of formula (I′) demonstrated robust pathway inhibition with 54 percent reduction of Axin2 upon treatment.

### Porcupine Inhibitor Compound of Formula (I′) in the Tight Skin Mouse Model (Tsk-1)

To further test the anti-fibrotic effects of compound of formula (I′), we turned to a mouse genetic fibrosis model, the tight skin model (Tsk-1). Tsk-1 mice contain a spontaneous duplication mutation of fibrillin-1 gene, which leads to activation of TGFβ signaling, and the subsequent fibrosis (increased in dermal and hypodermal thickness).

Five week old wild type or Tsk-1 mice were treated with or without compound of formula (I′) b.i.d. for 5 weeks (FIG. 3A). Similar to the results in the bleomycin model, compound of formula (I′) showed significant anti-fibrotic effects at all dose levels as measured by reduction of skin thickness, hydroxyproline content and myofibroblast count (FIG. 3B, FIG. 3C and FIG. 3D). The 5 and 10 mg/kg groups showed evidence of reversal of fibrosis in aforementioned readouts: FIG. 3B showed evidence that the skin thickness was reduced to levels lower than the baseline and closer to the control group (WT mice 10 week), FIG. 3C showed similar results concerning the hydroxyproline content levels and FIG. 3D showed also evidence of reduction of the myofibroblast count towards control level. As shown in FIG. 3F, treatment with compound of formula (I′) significantly reduced hypodermal skin thickness as demonstrated by H&E staining of mouse skin samples, which is the hallmark of fibrosis in Tsk-1 mice.

Similar to that described for the bleomycin-induced mouse fibrosis model, plasma exposures of compound of formula (I′) in this tight skin model were also determined following the last oral dose, and PK data are shown in FIG. 3E. Plasma exposures of compound of formula (I′) were comparable between the two separate experiments in two different models. And plasma exposures of compound of formula (I′) increased approximately dose proportionally from 2.5 mg/kg to 10 mg/kg.

### Combination of a wnt Inhibitor and a Second Active Iingredient:

The Wnt signaling pathway is known to have significant cross-talk with another key driver in fibrosis, the TGFβ signaling pathway. Reduction of the skin fibrosis markers has been observed in the clinical trial with anti-TGFβ antibody, Fresolimumab (Rice, L. M. et al. The Journal of clinical investigation 2015, 125, 2795-2807). In TGFβ signaling active mouse models including Tsk-1 model and adenovirus-based active TGFBR1 overexpressing model, inhibition of the Wnt pathway through overexpression of DKK1, strongly reduced Wnt signaling activities and attenuated TGFβ driven fibrosis, suggesting that Wnt signaling is the downstream effector of the TGFβ fibrosis (Akhmetshina, A. et al. Nature communications 2012, 3, 735). Consistently, when we treated mice with compound of formula (I′) and compound of formula (I″) in the Tsk-1 model, compound of formula (I′) and compound of formula (I″) demonstrated robust anti-fibrotic effects. Similarly, another Porcupine inhibitor, C59, could abrogate TGFβ-induced Axing induction in primary kidney fibroblasts (Madan, B. et al. Kidney international 2016, 89, 1062-1074). By targeting the main etiological drivers behind fibrosis concurrently, the TGFβ and Wnt signaling pathways, the combination of Porcupine inhibitor and TGFβ inhibitor is expected to be effective in the treatment of fibrosis, including SSc and SSS.

1. A method of treating scleroderma, comprising administering an effective amount of a wnt inhibitor of formula (I) 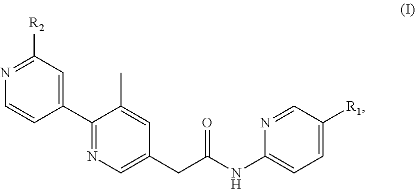
wherein
R₁ is 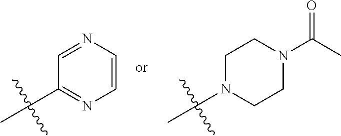
and R₂ is CH₃ or F. or a pharmaceutically acceptable salt thereof.

2. (canceled)

3. The method for the treatment of scleroderma according to claim 1, wherein the compound of formula (I) is selected from the group consisting of N-(5-(4-acetylpiperazin-1-yl)pyridin-2-yl)-2-(2′-fluoro-3-methyl-2,4′-bipyridin-5-yl)acetamide, and 2-(2′,3-dimethyl-2,4′-bipyridin-5-yl)-N-(5-(pyrazin-2-yl)pyridin-2-yl)acetamide, or a pharmaceutically acceptable salt thereof.

4. The method for the treatment of scleroderma according to claim 1, wherein the reversal of scleroderma is determined.

5. The method for the treatment of scleroderma according to claim 1, wherein the scleroderma is selected from skin systemic sclerosis, and stiff skin syndrome.

6. The method for the treatment of scleroderma according to claim 5, wherein the scleroderma is systemic sclerosis.

7. The method for the treatment of scleroderma according to claim 5, wherein the scleroderma is stiff skin syndrome.

8. (canceled)

9. The method for the treatment of scleroderma according to claim 1, wherein said wnt inhibitor is administered in treatment cycles comprising an administration period of up to 2 months, followed by a rest period.

10. The method for the treatment of scleroderma according to claim 9, wherein the rest period is at least one week to 3 months, preferably the rest period is from 1 to 4 weeks long.

11. The method for the treatment of scleroderma according to claim 9, wherein said wnt inhibitor is administered in treatment cycles comprising an administration period of up to 1 month

12. The method for the treatment of scleroderma according claim 9, wherein said wnt inhibitor is administered in treatment cycles comprising an administration period of up to 5 weeks .

13. The method for the treatment of scleroderma claim 9, wherein said wnt inhibitor is administered in treatment cycles comprising an administration period of up to 3 weeks.

14. The method for the treatment of scleroderma according to claim 9, wherein the rest period is 4 weeks.

15. The method for the treatment of scleroderma according to claim 3, wherein the wnt inhibitor is N-(5-(4-acetylpiperazin-1-yl)pyridin-2-yl)-2-(2′-fluoro-3-methyl-2,4′-bipyridin-5-yl)acetamide or a pharmaceutically acceptable salt thereof and is administered at a dose of 40 to 80 mg/day.

16. The method for the treatment of scleroderma according to claim 3, wherein said wnt inhibitor is 2-(2′,3-dimethyl-2,4′-bipyridin-5-yl)-N-(5-(pyrazin-2-yl) pyridin-2-yl)acetamide or a pharmaceutically acceptable salt thereof and is administered at a dose of 5 to 50 mg/day.

17. (canceled)

18. The method for the treatment of scleroderma according to claim 1, wherein said wnt inhibitor or a pharmaceutically acceptable salt thereof is administered in combination with a second active ingredient.

19. The method for the treatment of scleroderma according to claim 18, wherein the second active ingredient is an inhibitor of the TGFbeta signaling pathway.

20. The method for the treatment of scleroderma according to claim 19, wherein the second active ingredient is an inhibitor of the TGFbeta signaling pathway selected from fresolimumab and metelimumab.

21. The method for the treatment of scleroderma according to claim 18, wherein the second active ingredient is the inhibitor of an activin receptor type 2B.

22. The method for the treatment of scleroderma according to claim 21, wherein the second active ingredient is selected from bimagrumab, ACE-031, LY2495655 and PF-06252616.

23. The method for the treatment of scleroderma according to claim 22, wherein the second active ingredient is bimagrumab.

24. A pharmaceutical combination for a method of treating scleroderma comprising a wnt inhibitor of formula (I) 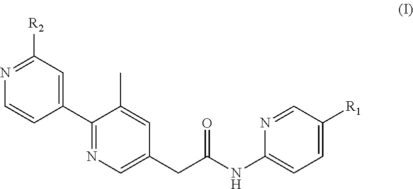
and a second active ingredient.

25. (canceled)

26. The pharmaceutical combination according to claim 24, wherein the wnt inhibitor is selected from the group consisting of N-(5-(4-acetylpiperazin-1-yl)pyridin-2-yl)-2-(2′-fluoro-3-methyl-2,4′-bipyridin-5-yl)acetamide, a pharmaceutically acceptable salt thereof, and 2-(2′,3-dimethyl-2,4′-bipyridin-5-yl)-N-(5-(pyrazin-2-yl)pyridin-2-yl)acetamide, or a pharmaceutically acceptable salt thereof.

27. The pharmaceutical combination according to claim 24, wherein the second active ingredient is selected from fresolimumab, metelimumab, bimagrumab, ACE-031, LY2495655 and PF-06252616.

28. The pharmaceutical combination of claim 27, wherein the second active ingredient is bimagrumab.

29. The pharmaceutical combination of claim 24, wherein the wnt inhibitor and the second active ingredient are administered separately or together.

30. The pharmaceutical combination of claim 24, wherein the wnt inhibitor and the second active ingredient are administered independently at the same time or separately within time intervals.

31. (canceled)

32. (canceled)

33. The pharmaceutical combination according to claim 24, wherein scleroderma is selected from systemic sclerosis, and stiff skin syndrome.

34. The pharmaceutical combination according to claim 33, wherein scleroderma is systemic sclerosis.

35. The pharmaceutical combination according to claim 33, wherein scleroderma is stiff skin syndrome.

36. (canceled)

37. (canceled)

38. (canceled)

39. The method for the treatment of scleroderma according to claim 1, wherein the wnt inhibitor is 2-(2′,3-dimethyl-2,4′-bipyridin-5-yl)-N-(5-(pyrazin-2-yl)pyridin-2-yl)acetamide or a pharmaceutically acceptable salt thereof.
